(19) United States
(12) Patent Application Publication
(10) Pub. No.: US 2006/0122468 A1
(43) Pub. Date: Jun. 8, 2006

(54) Nutritional counseling method and server

(75) Inventor: Mali Tavor, Tel Aviv (IL)

Correspondence Address: Martin MOYNIHAN;c/o ANTHONY CASTORINA, SUITE 207, 2001 JEFFERSON DAVIS HIGHWAY, ARLINGTON, VA, 22202, US

(73) Assignee: Mali Tavor Ltd.

(21) Appl. No.: 10/986,764

(22) Filed: Nov. 15, 2004

(51) Int. Cl. A61B 5/00

(52) U.S. Cl. 600/300; 600/921

(57) ABSTRACT

A method for nutritional counseling consists of the following steps. First, a user is allowed to connect remotely over a network. Next, baseline personal information is received from a user. The user is then provided with nutritional guidelines over the network connection in accordance with the baseline personal information. Once the nutritional guidelines are established, food consumption information is received over the network connection from the user, and preferably logged into a food diary. The received food consumption information is analyzed in accordance with the nutritional guidelines, and dietary feedback is provided to the user over the network connection in accordance with the performed analysis.

### FIELD AND BACKGROUND OF THE INVENTION

The present embodiments relate to providing a user with a method for nutritional counseling over a network connection and, more particularly, to automatically providing the user with nutritional guidelines, monitoring the user's adherence to the guidelines, and providing feedback to the user accordingly.

The problems of overweight and obesity have reached epidemic proportions globally, with more than 1 billion adults overweight, 300 million of them clinically obese. These problems are a major contributor to the global burden of disease and disability. Approximately 127 millions (65 percent) adults in the U.S. are overweight and 60 millions (30 percent) are obese. Obesity has increased for both genders over the last decade. Obesity has serious social and psychological dimensions, and affects virtually all ages and socioeconomic groups.

Obesity is commonly determined by a comparison of an individual's body weight and height to some standard of acceptable or desirable weight. Desirable weight standards are derived in a number of ways.

One commonly used weight standard is based on a mathematical formula known as the body mass index (BMI), which represents weight levels associated with the lowest overall risk to health. The BMI is calculated as:

$$BMI = \frac{Weight ( kg . )}{{[ Height ( meters ) ]}^{2}} \qquad ( 1 )$$

The BMI is more highly correlated with body fat than other indicators of height and weight. Individuals with a BMI of 25 to 29.9 are considered overweight, while individuals with a BMI of 30 or more are considered obese. All adults who have a BMI of 25 or more are considered at risk for premature death and disability as a consequence of overweight and obesity. These health risks increase as the severity of an individual's obesity increases.

A second weight standard is based on height and weight data collected on a representative group of the U.S. population in the National Health and Nutrition Examination Survey (NHANES), a survey by the U.S. National Center for Health Statistics. Another desirable weight table was created by the Metropolitan Life Insurance Company, based on their client population.

A high percentage of body fat is induced by a combination of genetic, environmental and psychological influences. Evidence for a strong genetic contribution of human obesity comes from a variety of sources. Twins and familial aggregation studies suggest that genetic factors account for 60% to 80% of the predisposition to obesity. In general, genes that influence body weight encode the molecular components of the physiological system that regulates body weight. Studies have shown that metabolic rate, the thermic effect of food, and spontaneous physical activity show genetic components. There are at least several dozen genes involved in obesity, and not only does current weight status have an inherited component, but the metabolic processes underlying weight gain may have strong genetic influences.

The genetic expression of obesity now occurs in an environment that permits the expression of the propensity to store body fat. Since the prevalence of obesity has risen in the last decades, it is seen that despite the importance of genetics in determining predisposition to obesity, development of obesity itself is strongly influenced by environmental triggers. Environmental triggers include an overall decrease in physical activity and, therefore, total energy expenditure. At the same time, there has been an increase in food availability. With the advent of fast food and convenience foods, there has also been an increase in the caloric density of readily available foods, coupled with aggressive and sophisticated food marketing via the mass media. In addition, there has been a significant increase in the portion size of foods that are sold and served Our lifestyle has become increasingly more stressful limiting the time and emphasis on food planning and preparation, often leading to chaotic patterns of eating.

As the environment promotes increased caloric consumption and decreased physical activity, Western culture places a value on attractiveness and interprets slenderness as essential to attractiveness. This interpretation of attractiveness demands conformity to a narrow definitions and those who fail to conform are expected to be denied success, love, power and other rewards. Thinness can be a motivator to change eating and exercise behaviors but the changes are most often extreme and short-lived. The consequences are usually a rapid weight loss, and then regaining of weight to an even higher level.

Mood state and weight status are frequently related. Surveys show that depression plays a substantial role in the patterns of weight changes in adults. Studies have indicated that brain serotonin is critically involved in controlling mood, and many people learn they can transiently reduce depressive symptoms by eating.

Obesity and overweight pose a major risk for serious diet-related chronic diseases, including diabetes, cardiovascular disease, hypertension and stroke, certain forms of cancer, musculoskeletal disorders, sleep apnea, and gallbladder disease. Consequences range from increased risk of premature death, to serious chronic conditions influencing the overall quality of life.

The Center for Disease Control and Prevention (CDC) estimates that the U.S. spends as much as 75 billions a year in medical expenditures related to obesity. This finding is a dramatic illustration of the devastating economic impact obesity has on health care systems.

Body weight is tightly regulated. An increase in fatty tissues results from the failure of this sophisticated regulatory system that balances total energy intake and expenditure. Obesity is the result of a positive energy balance due to excessive food intake and/or inadequate energy expenditure.

From measurements of the nitrogen balance it has long been known that the body tends to maintain its protein balance both spontaneously and effectively. Hence, weight regulation is the result of interactions between the metabolism and the intake of carbohydrates and fats. Since glycogen reserves are typically 100 times lower than lipid reserves, and since maintaining the blood glucose level is particularly important for brain function, biological evolution has been forced to develop control mechanisms that give higher priority to maintaining the carbohydrate balance than to maintaining the fat and energy balances. It is clear that the primary role of the enzymatic and hormonal control mechanisms, and more particularly that of insulin, is to maintain the level of blood glucose and to adjust the level of glucose oxidation to carbohydrate input. The rapid increase in the proportion of glucose in the mixture of substrates oxidized after a meal illustrates the efficacy of these mechanisms, a fundamental feature of which is that the body can adapt to highly variable levels of carbohydrate intake. This adjustment at the metabolic level is complemented by the effect of the body's carbohydrate reserves on food intake.

Hunger triggered by a drop in the blood glucose level or glycogen reserves is a well-known phenomenon. The mechanisms that limit the intake of food, on the other hand, are more difficult to pin down. That they play a role, however, is implied by the fact that even when access to a wide variety of foods is unrestricted, glycogen reserves are spontaneously maintained well below the level that would trigger a marked transformation of carbohydrates into fat.

Glucose oxidation is effectively curtailed during carbohydrate deprivation, so that the small amounts of glucose formed by gluconeogenesis from amino acids and from triglyceride-glycerol are sufficient to sustain minimal blood glucose levels. Consuming carbohydrate to maintain glucose availability reduces the need to obtain energy by amino acid oxidation. Ingestion of some 100 g of carbohydrate per day reduces nitrogen excretion by about half, a phenomenon well known as the protein-sparing effect of dietary carbohydrate.

The activation of pyruvate dehydrogenase by insulin is one of the mechanisms allowing insulin to promote carbohydrate oxidation. The first irreversible steps in the degradation of the branched-chain amino acids are catalyzed by alpha-ketoacid oxidizing enzyme complexes, which are similar to pyruvate dehydrogenase and can also be activated by insulin. It is thought that glucose availability and the readiness to oxidize pyruvate, which is enhanced by insulin, explain the carbohydrate-sparing effect of exogenous carbohydrate. However, high insulin levels also open the pathway for the irreversible degradation of the Branched Chain Amino Acids (BCAA), notably in skeletal muscle. Since the BCAA are indispensable amino acids, their loss by the irreversible oxidation of their alpha-keto derivatives causes the other amino acids to be degraded as well, since they cannot be used for protein synthesis without enough BCAA.

The presence of fat in a meal affects the mixture of substrates oxidized in the following hours and days only marginally. In contrast to proteins and carbohydrates, the oxidation of fatty acids is not adjusted directly in response to variations in the amount consumed. Consuming foods with a high fat content rapidly leads to accumulation of fat in the adipose tissue. When the proportion of fat in the mix of substrates oxidized in the body is the same as the proportion of fat in the diet, the mechanisms that adjust food intake in response to the body's carbohydrate requirement can also maintain the balance between intake and oxidation of dietary fats. Maintaining this balance allows the composition of the body to stabilize.

Dietary fat is also more efficient in its metabolic usage than carbohydrate and protein. Some of the adenosine triphosphate (ATP) generated by substrate oxidation serves to replace the ATP spent for the transport, activation, recycling, regeneration, and excretion of the substrates being oxidized. Based on current knowledge of metabolic pathways, net ATP yields can be estimated. These seem to be higher for fat (90%) than for carbohydrate (75%) or for protein (60%). The difference in the net ATP yields from carbohydrate and fat is due to the greater cost for the conversion of dietary carbohydrate to glycogen than for storing triglycerides, and because additional ATP is expended for the reconversion of lactate to glucose in the Cori cycle. During the conversion of glucose to fat, 25% of the energy that is channeled into the lipogenic pathway is used for the synthesis of the ATP expended for fatty acid synthesis.

The size that the adipose tissue mass has to reach for fat oxidation rates to become commensurate with fat intake is determined by inherited regulatory features that influence the relative rates of glucose and fatty acid oxidation, dietary fat intake, physical activity, and by the range within which habitual glycogen levels are maintained. These factors may explain why a particular degree of fatness tends to remain established when dietary and lifestyle conditions are constant, explaining the stability of body weight, and postulating the existence of a particular set-point. Several studies have shown that adjustment of fat oxidation to increased fat consumption is more sluggish in individuals who are considered to be predisposed to obesity.

Physical activity is effective in preventing or limiting excess fat accumulation, although it is not yet clear whether the influence of physical activity is due merely to its effect in increasing energy turnover or whether it is mediated in part by promoting fat oxidation toga greater extent than glucose oxidation. Furthermore, a lifestyle that sustains fitness seems to afford some protection against fat gains during occasional transition from low- to high-fat intakes.

Skeletal muscle readily uses fatty acids for energy production. During exertion substrate oxidation in the muscle mass can increase twenty-fold. In the post-absorptive state, fatty acids are the primary oxidized fuel in muscle, whereas during exertion, great demands are initially placed on the glycogen reserve of the muscles, with a subsequent shift toward increasingly greater use of fatty acids, mobilized from muscle fat stores and adipose tissue.

According to the NHANES, obesity prevalence among men and women has grown substantially (by a factor of 2-3) in the last three decades. The most profound growth in obesity is in the relatively young age group of 20-39. Obesity prevalence among adolescents has also grown threefold. Overweight and obesity increases steadily with age for both men and women. At a minimum, half of all age groups are overweight and at least 20 percent are overweight. Obesity has increased across all education levels, and is higher among population of low education level.

Weight management is a major focus of personal, social, and economic activity in many countries. Consumer surveys in the U.S. show that 48% of adult Americans consume diet soft drinks, 34% consume sugar-free frozen desserts and ice cream, and 30% consume sugar-free gum. Reduced-fat products are also widely used. 56% of adult Americans consume low fat milk, 49% consume reduced-fat dairy products, 46% consume reduced-fat salad dressings, and 33% consume reduced-fat snacks.

The American Dietetic Association's (ADA) 2002 nutrition trends survey shows that 38% of Americans claim they are already trying to control their weight. They say that they have made significant adjustments in their eating behavior during the past two years to achieve a healthy diet. This is compared to 28% in the year 2000. 30% of those-surveyed claim that they know they should modify their behavior, but for some reason haven't done so. This is compared to 40% at the year 2000. 32% are not interested in better eating behavior, as compared to 40% in 1997.

The reasons cited by adult Americans who need to lose weight are varied. 50% think they fail because they don't exercise enough. 36% think they too often splurge on favorite foods. 33% think they snack too much. 28% think they eat too many high fat foods. 28% think they often eat for emotional reasons. 26% think they overeat at mealtimes. 23% think they have trouble eating properly in restaurants.

A wide variety of dieting and nutritional services are available to individuals. Dietitians are available to address individual patients weight concerns, provide diet: suggestions, and monitor patient progress. However, the patient's access to the dietitian is limited. The dietitian provides guidance and reinforcement only during relatively infrequent visits, and is not available for consultation on a daily basis. Many individuals require more ongoing support to maintain a nutritional program.

One way of providing a dieter with ongoing support is by an online service. One such service is provided by the well-known Weight Watchers organization, which traditionally focused on subscriber attendance at support meetings. Weight Watchers provides an online service to subscribers of their Flex Plan. The Flex Plan is based on a points system. Each food is awarded a number of points, and the dieter is expected to eat a certain number of points each day. The online Points Tracker has the subscriber record what he or she has eaten throughout the day or week, and provides a point count of the foods eaten. However, the Points Tracker has limited responses. Although the points are tracked, a thorough analysis of user food consumption is not performed, and the user is not provided with recommendations for diet management.

In Pat. Appl. No. 20030165799, Bisogno presents a computer program, method, and system for dynamically and interactively providing nutrition content information for consumables such that a user may monitor, tailor, plan, and review their intake thereof in light of a health-related interest or concern, such as, for example, weight-loss, food allergies, or diabetes or other nutrition affected illnesses or disabilities. Consumables are categorized and displayed in lists associated with an appropriate color to draw attention to relative nutrition content and to facilitate quicker and easier evaluation of a consumable of interest. Summaries are provided of a user's actual intake in light of a pre-established target intake for a particular day. Detailed reports may be generated showing consumption over a user-specifiable time period. Bisogno's system is primarily an informational tool, for self-monitoring by the user. In Bisgno's system the user defines his own health and diet guidelines, and uses the nutritional database in order to ensure that he stays within these guidelines. The user must have a predetermined diet strategy before utilizing Bisogno's system.

In US Pat. Appl. No. 2003/0208383, Hauck et al. present a system which helps a user track nutritional intake values, and assists the user in maintaining nutritional intake goals. The disclosure teaches looking up of the nutritional values of foods, adding foods or food combinations to a temporary memory, maintaining a daily diary of nutritional intake, generating the average nutritional intake for a selected number of days, and storing nutritional goals. In addition, the disclosure allows the user to create custom meal totals, representing user-included food items and their corresponding nutritional values that the user consumes in a meal. Further, the disclosure compares nutritional goals with the averages of actual nutritional intake. However, Hauck provides only a user tool for monitoring food intake, and does not provide active diet management.

Pat. Appl. 20030225731 Vidgen provides a dietary menu planning system. The dietary menu planning system receives personal characteristics and food preferences for an individual, including a desired physiological rate of change for the individual. Based on the physiological rate of change and other personal characteristics, a set of dietary constraints is developed for the individual. A dietary menu plan is created for the individual including food items selected by the individual and with quantities determined to maximize the food preferences of the individual while satisfying the dietary constraints and to maximize the user's food preferences. Vidgen's system provides recommended menus, but does not provide the user with any tools for maintaining the recommended diet.

Nugent, D., K. Dube, and B. Wu. present a dietary analysis system in the publication “DAAS: A Web-Based System for User-Specific Dietary Analysis and Advice for the Public Healthcare Domain.”, Exploring the New Generation Computing Technology: Proc of the International Conference for Young Computer. Scientists (ICYCS 2003), 8-10 August, Harbin, China, World Publishing Corporation, China, pp. 461-464. Nugent et al. present a system, which elicits dietary and lifestyle information from a user, analyses the elicited information, and determines the appropriate advice to give to the user w.r.t diet and lifestyle improvements. The Dietary Analysis and Advice System (DAAS) is designed to capture diet and nutritional domain knowledge, use a knowledge representation formalism to capture and exploit the knowledge in analyzing a user's diet and lifestyle, generate the relevant advice; and present the advice to the user using a web-based mechanism. Again, the DAAS system provides the user with a dietary analysis, but fails to include any mechanism to ensure that the user implements the analysis results.

The above described nutritional-aid tools all fail to provide a user with effective support over the entire path towards achieving his nutritional goals. There is thus a widely recognized need for, and it would be highly advantageous to have, a nutritional counseling system devoid of the above limitations.

### SUMMARY OF THE INVENTION

According to a first aspect of the present invention there is provided a method for nutritional counseling which consists of the following steps. First, a user is allowed to connect remotely over a network. Next, baseline personal information is received from a user. The user is then provided with nutritional guidelines over the network connection in accordance with the baseline personal information. Once the nutritional guidelines are established, food consumption information is received over the network connection from the user, and preferably logged into a food diary. The received food consumption information is analyzed in accordance with the nutritional guidelines, and dietary feedback is provided to the user over the network connection in accordance with the performed analysis.

Preferably, the food consumption information consists of a type and quantity of food eaten, and the time and date the food was eaten.

Preferably, the method contains the further step of logging the food consumption information in a food diary.

Preferably, the method contains the further step of receiving current personal information from the user.

Preferably, the method contains the further step of modifying the nutritional guidelines in accordance with current and previously-provided personal information.

Preferably, the guidelines are for maintaining a balance of nutritional components consumed by the user.

Preferably, the nutritional components consist of proteins, carbohydrates, and fats.

Preferably, the analysis is in accordance with nutritional information stored for a plurality of foods in a foods database.

Preferably, the method contains the further step of updating the foods database to include nutritional information for a user-specified food.

Preferably, the personal information consists of the user's gender, age, current weight, height, and nutritional goals.

Preferably, the personal information includes the user's physical activity regimen.

Preferably, the method contains the further step of providing the user with physical activity guidelines in accordance with the personal information, over the network connection.

Preferably, the personal information includes the user's health status.

Preferably, nutritional guidelines are established by calculating a recommended weight range in accordance with the user's gender, height, and age, determining a recommended caloric intake level in accordance with the user's gender, height, weight, age, physical activity level and nutritional goals, and determining respective allowances of proteins, carbohydrates, and fats, in accordance with the recommended caloric intake level.

Preferably, the analysis consists of determining a list of foods eaten by the user during a specified time period, calculating the total quantities of carbohydrates, proteins, and fats for foods on the list, and comparing the total quantities to the nutritional guidelines.

Preferably, the feedback includes the remaining daily allowances of the nutritional components.

Preferably, the feedback includes alerting the user to non-adherence to the guidelines.

Preferably, the feedback includes a nutritional error notification, and suggestions for correcting said nutritional errors.

Preferably, the network consists of at least one of: the Internet, a Public Switched Telephone Network (PSTN) a cellular telephone network, a cable TV network, a satellite TV network, a local-area network (LAN), a wide-area network (WAN), and a metropolitan-area network (MAN).

Preferably, the method contains the further step of providing the user with a recommended menu.

Preferably, the analysis is in accordance with physical activity information received from the user.

Preferably, the method contains the further step of monitoring a user's physical activity, in comparison to a physical activity level provided with the personal information.

Preferably, the method contains the further step of monitoring a user weight in accordance with weight information received from the user.

Preferably, the method contains the further step comparing a user weight to an expected user weight.

Preferably, the method contains the further step of calculating the portions of carbohydrates, proteins, and fats present in a specified food type and quantity.

Preferably, the method contains the further step of receiving a recipe consisting of a listing of types of foods and respective quantities, and translating the recipe into quantities of carbohydrates, proteins, and fats.

Preferably, the method contains the further step of billing the user.

According to a second aspect of the present invention there is provided a nutritional counseling server, consisting of a user interface, a guideline establisher, a logger, and a nutrition analyzer. The user interface interfaces to a user over a network. The guideline establisher obtains baseline personal information from the user and establishes nutritional guidelines for the user in accordance with the personal information. The logger receives food consumption information received from the user and logs the food consumption information in a food diary. The nutrition analyzer analyzes the food consumption information according to the guidelines, and provides dietary feedback to the user in accordance with the analysis.

Preferably, the nutritional counseling server further contains a foods database associated with the nutrition analyzer, for storing nutritional information for a plurality of foods.

Preferably, the foods database is updateable by the user to include nutritional information for a user-specified food.

Preferably, the the guidelines establisher consists of a weight determiner, for calculating a recommended weight range in accordance with the user's gender, height and age, an energy calculator, for determining a recommended caloric intake level in accordance with the user's gender, height, weight, age, physical activity level and nutritional goals, and a guideline specifier associated with the weight determiner and the energy calculator, for determining a respective recommended allowance of proteins, carbohydrates, and fats, in accordance with the recommended caloric intake level.

Preferably, the the nutritional analyzer consists of a food list generator, for retrieving a list of foods eaten by the user during a specified time period from the food diary, a quantity summer associated with the food list generator, for calculating the total quantities of carbohydrates, proteins and fats for the list of foods, and a comparator, for comparing the total quantities to the nutritional guidelines.

Preferably, the feedback consists of at least one of the following types of recommendations: a recommended menu, a recommended daily allowance of the nutritional components, a remaining daily allowance of the nutritional components, nutritional error notification, and suggestions for correcting nutritional errors.

Preferably, the nutritional counseling server further contains a physical activity analyzer .associated with the nutrition analyzer, for calculating energy expenditure in accordance with physical activity information received from the user.

Preferably, the nutrition analyzer-provides interactive responses to user entries.

Preferably, the network consists of at least one of: the Internet, a PSTN, a cellular telephone network, a cable TV network, a satellite TV network, a LAN, a WAN, and a MAN.

Preferably, the nutritional counseling server further contains a menu planner associated with the foods database, for providing a recommended menu in accordance with the guidelines.

Preferably, the logger is further operable to record weight information received from the user in the diary.

Preferably, the logger includes a meal entry unit for user entry of a meal time, foods eaten during the meal, and respective quantities of the foods eaten.

Preferably, the nutritional counseling server further contains a food calculator, for calculating quantities of carbohydrates, proteins, and fats present in a specified food type and quantity.

Preferably, the food calculator includes a recipe calculator, for translating a specified recipe into quantities of carbohydrates, proteins, and fats.

Preferably, the nutritional counseling server further contains billing functionality.

Preferably, the nutritional counseling server further contains a favorite food list for recording the user's favorite foods for incorporation into a recommended menu.

According to a third aspect of the present invention there is provided a computer-readable storage medium containing a set of instructions for nutritional counseling, consisting of a personal information input routine, a guideline establishment routine, a consumption information input routine, an analysis routine, and a feedback routine. The personal information input routine is for inputting personal information from a user. The guideline establishment routine is for establishing nutritional guidelines for the user in accordance with the personal information and outputting the, guidelines to the user. The consumption information input routine is for inputting food consumption information from the user. The analysis routine is for analyzing the received food consumption information in accordance with the guidelines. The feedback routine is for outputting dietary feedback information to the user in accordance with the results of the analysis.

The present invention successfully addresses the shortcomings of the presently known configurations by providing a method and server for nutritional counseling which provides a user with nutritional guidelines, monitors the foods eaten by the user, and provides the user with nutritional feedback accordingly. The connection with the user is provided over a network. Preferably the interaction with the user is interactive, so that the user can get immediate feedback to the nutritional and personal information he provided.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified flowchart of a method for nutritional counseling, according to a preferred embodiment of the present invention;

FIG. 2 shows an example of a personal information input screen, for user input of the personal information;

FIG. 3 shows an example of nutritional recommendations provided to the user, on the basis of received personal information.

FIG. 4 shows a recommended weight loss table which is preferably displayed to the user indicating the recommended change in the user's weight over time;

FIGS. 5a and 5b show an examples of food-entry screens displayed to the user for the purpose of entering foods into the food diary;

FIG. 6a shows atypical feedback screen which is displayed to the user after the user finishes entering food consumption information for a given meal;

FIG. 6b shows a typical feedback screen which is displayed to the user at the end of the day;

FIG. 7 is a simplified flowchart of a method for establishing nutritional guidelines, according to a preferred embodiment of the present invention;

FIG. 8 is a simplified flowchart of a method for analyzing received food consumption information, according to a preferred embodiment of the present invention;

FIG. 9a is a simplified block-diagram of a nutritional counseling server, according to a preferred embodiment of the present invention;

FIG. 9b shows an example of a new food entry screen, for user addition of a food to the foods database;

FIG. 10 shows an example of a data entry screen for the food calculator;

FIG. 11 is a simplified block diagram of a guidelines establisher, according to a preferred embodiment of the present invention;

FIG. 12 is a simplified block diagram of a nutritional analyzer, according to a preferred embodiment of the present invention.

### DESCRIPTION OF THE PREFERRED EMBODIMENTS

The present embodiments are of a method and system for nutritional counseling. Specifically, the present embodiments establish nutritional guidelines for the user, monitor the foods eaten by the user, and provide the user with nutritional feedback.

The present embodiments are based on the notion that successful weight management for improving overall health requires a lifelong commitment to healthful lifestyle behaviors, emphasizing sustainable and enjoyable eating practices and daily physical activity. Although the knowledge base has greatly expanded regarding the complex causation of increased body fat, little progress has been made in long-term maintenance intervention. Lifestyle modifications in food intake and exercise remain the hallmarks of effective treatment, but are difficult to initiate and sustain over the long term.

Any changes in dietary intake and exercise pattern which decrease caloric intake below energy expenditure will result in weight loss. However, recommended changes should be directed toward the long-term maintenance of the desired weight, and improved physiological and psychological health. Currently, available data on lifestyle weight loss interventions indicate that they produce low levels of sustained loss. Typically reported weight losses remaining after 4-5 years are about 3% to 6% of initial body weight.

Dietary and physical activity modifications need to be integrated and accepted as away of life. Improvements in weight loss maintenance are shown when the risks of relapses are addressed as part of the intervention, while emphasizing long-term changes in eating and physical activity behaviors. It has also been shown that long-term treatment programs and follow-up programs are effective in preventing relapses.

In light of the above, the present embodiments focus on two main elements of nutritional counseling:

1) Physiological principles: teaching the user the physiological principles of long-term weight maintenance, based on scientific knowledge concerning body usage of the different energy sources, and by adjusting food intake and physical activity so that the excess body fat is burnt effectively and the desired body weight is maintained easily. The present embodiments use scientific formulas for adjusting the proper eating pattern for each patient. The difference between the fat loss regime and the weight maintenance regime lies in the recommended caloric balance, which is negative during the fat loss and balanced for weight maintenance.

2) Behavioral principles: teaching the user how to translate the physiological principles to a comfortable and enjoyable way of life for the long run.

The principles and operation of a method and server for nutritional counseling according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For purposes of example, the following embodiments are based on guidelines developed according to the nutritional principles given below. The following principles are for illustration only, and are not limiting.

1) Total energy intake: for weight maintenance the recommended energy intake is balanced with expected energy expenditure, and for weight reduction the balance is negative (about 700 calories less than expected energy expenditure).

2) Carbohydrates intake. 55-60% of the energy intake (in order to maintain the blood glucose level for brain function, to prevent hunger or lack of control over eating, and to minimize gluconeogenesis from amino acids for sparing muscle mass)

3) Protein intake: 1 gram per kilogram body weight (in order to maintain normal building processes in the body)

4) Fat intake: 20-25% of the energy intake (in order to prevent accumulation of fat in the adipose tissue)

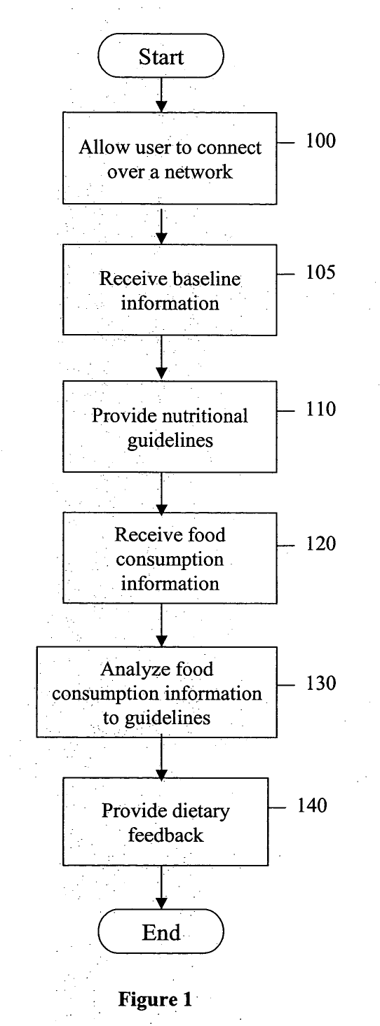

Reference is now made to FIG. 1, which is a simplified flowchart of a method for nutritional counseling, according to a preferred embodiment of the present invention. In step 100, a user is allowed to connect over a network. In the preferred embodiment, the network is one of: the Internet, a Public Switched Telephone Network (PSTN), a cellular network, a cable TV network, a satellite TV network, a local-area network (LAN), a wide-area network (WAN), and a metropolitan-area network (MAN).

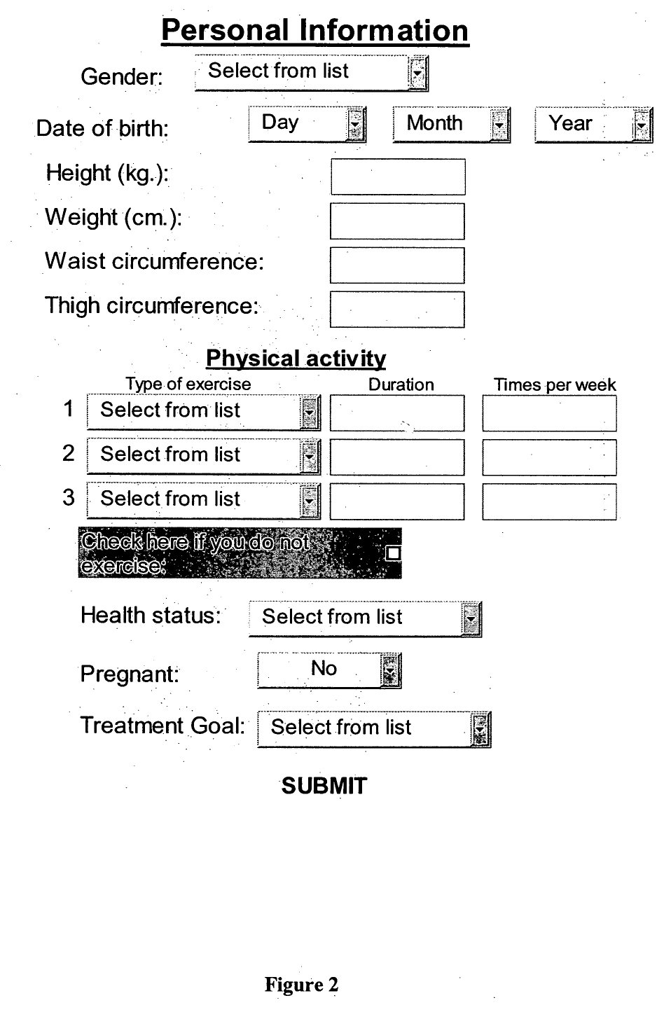

In step 105, baseline personal information is received from the user over the network. The user inputs all the data needed by the system, preferably including: gender, date of birth or age, current weight, height, physical activity regimen, and/or health status. The user preferably also determines his or her goals, such as a diet for reducing weight, for maintaining a current weight, for enhancement of muscle mass, for pregnancy, or for lactation. FIG. 2 shows an example of a personal information input screen, for user input of the personal information.

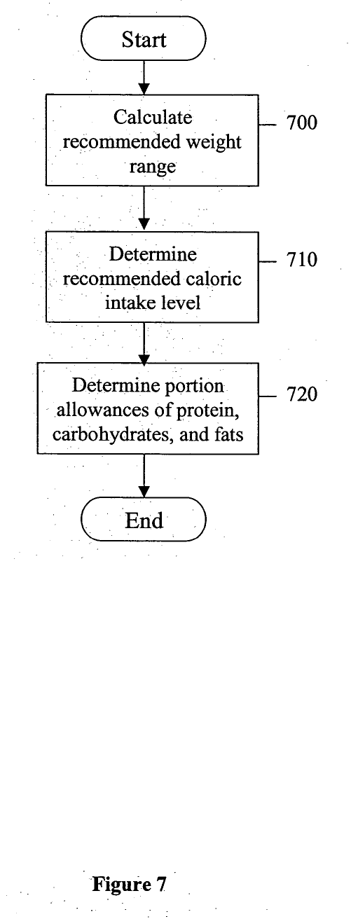

In step 110, nutritional guidelines are provided to the user over the network, based on the baseline personal information obtained in step 105. The baseline information is used to calculate nutritional recommendations for the given user. At the end of this stage the system recommends a suitable dietary pattern for reaching the user goals. In the preferred embodiment, a suitable caloric level is determined and the the nutritional recommendations provided to the user are adjusted so as to maintain a balance of nutritional components in the user's diet. Preferably, the nutritional components consist of proteins, carbohydrates, and fats, so that changes in the glycogen reserves do not influence food intake. A preferred embodiment of the process of establishing nutritional guidelines is presented below for FIG. 7.

With the nutritional guidelines, the user is preferably provided with a summary of his current nutritional behavior and/or appropriate comments and general nutritional information. The summary may contain information regarding his current calorie expenditure, a recommended weight range, and an expected time frame for reaching his nutritional goals. Preferably, the baseline information is also used to determine physical activity guidelines for the user, specifying a recommended physical activity regimen. In the preferred embodiment, the user is provided with a summary of his physical activity status, for example whether he exercises enough and whether the type and quantity of exercise result in a significant increase in his daily portion allowances, and suggested corrections or changes to his current physical activity regimen.

The allowances of the nutritional components may be specified by various measures. The allowances may be specified by weight or with any other defined unit. The present embodiments are directed at specifying the allowances of proteins, carbohydrates, and fats in portions, where the portions consist of:
1 portion of carbohydrates=15 grams of carbohydrates and 2 grams of protein
1 portion of proteins=10 grams of proteins and 5 grams of fats
1 portion of fats=5 grams of fats

Preferably, a physical activity regimen is also provided. For example, the user may be recommended to perform weak aerobic workout for at least 30 minutes three or more times week. During exertion, substrate oxidation in the muscle mass may increase twenty-fold. There is a great initial demand on the glycogen reserve of the muscles, with a subsequent shift toward increasingly greater use of fatty acids, mobilized from muscle fat stores and adipose tissue. Also, without exercise, loss of lean body mass with dieting alone contributes to a decrease in metabolic rate. Since skeletal muscle readily uses fatty acids for energy production this decrease diminishes fat oxidation.

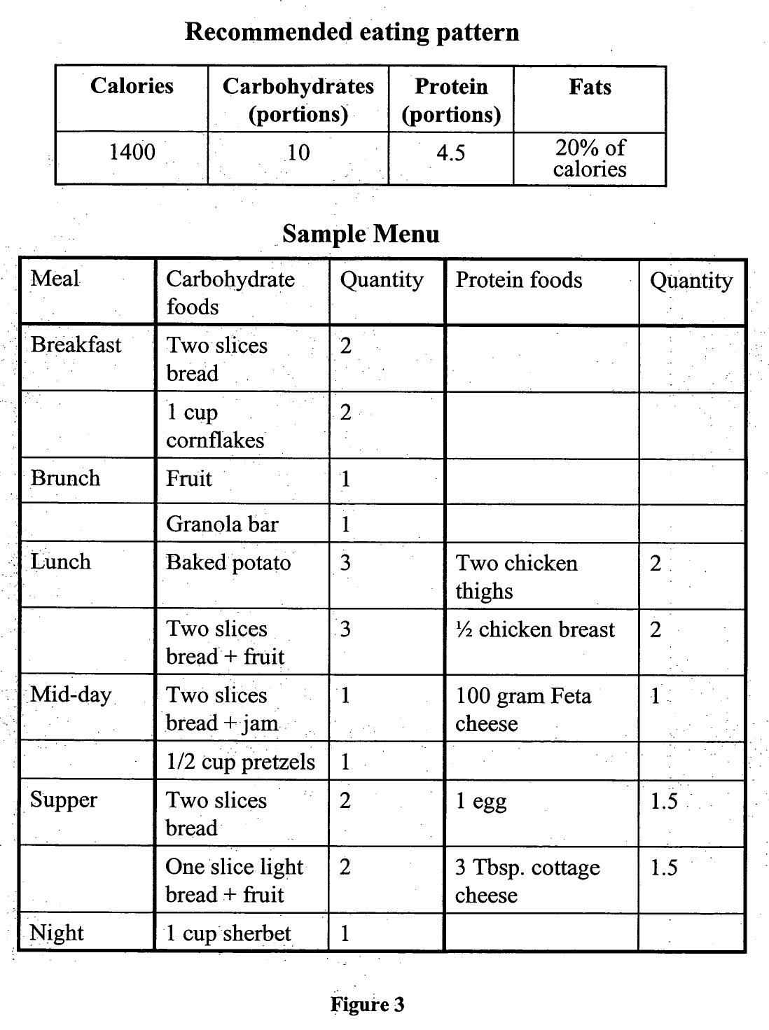

FIG. 3 shows an example of nutritional recommendations provided to the user in step 110, on the basis of the baseline information input during step 105. The user is given a recommended allowance of calories, carbohydrates, protein, and fat. The carbohydrate and protein allowances are specified in portions, and the fat allowance is given as a percentage of the calorie allowance. The user is also provided with one or more sample menus, illustrating how the recommended allowances may be translated into a sequence of meals and snacks during the day.

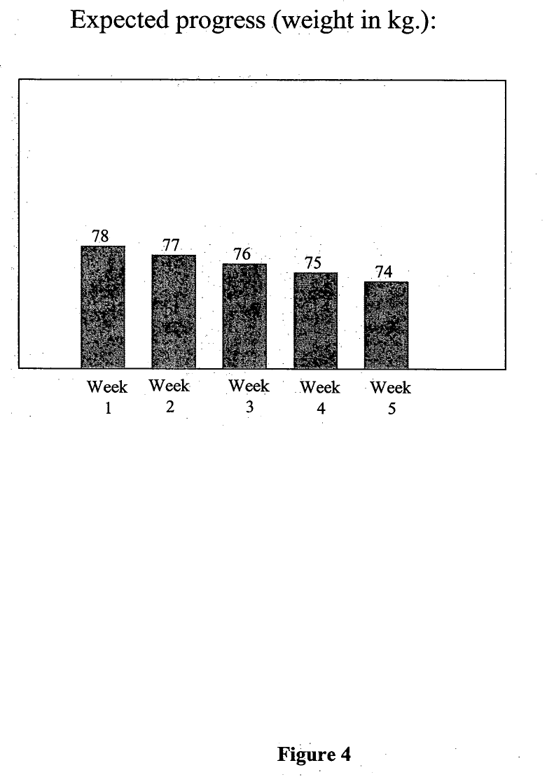

FIG. 4 shows a weight loss table which may be displayed to the user indicating the recommended change in the user's weight over time. Preferably, the recommended weight loss per week is 0.5-1 Kg.

In step 120, food consumption information is received from the user over the network. The user periodically inputs a list of foods eaten, which are checked against the user's nutritional recommendations. The food consumption information preferably gives the time the food was eaten, the food or foods eaten at the specified time, and the quantity of food eaten. In the preferred embodiment, the food consumption information is logged in a food diary.

The foods may be entered in various ways, for example by choosing a certain food from a list or by inserting nutritional values from a food label (for a food not present in the system database). The quantities may also be specified variously, for example by unit or by weight.

Preferably, nutritional information for specified foods is stored in a foods database. The nutritional information preferably includes the calorie, protein, carbohydrate and fat contents of various foods. In the preferred embodiment, the food database is updateable by the user. The user may thus add a purchased or homemade food to the foods database. User-specified foods can then be given as part of the food consumption information.

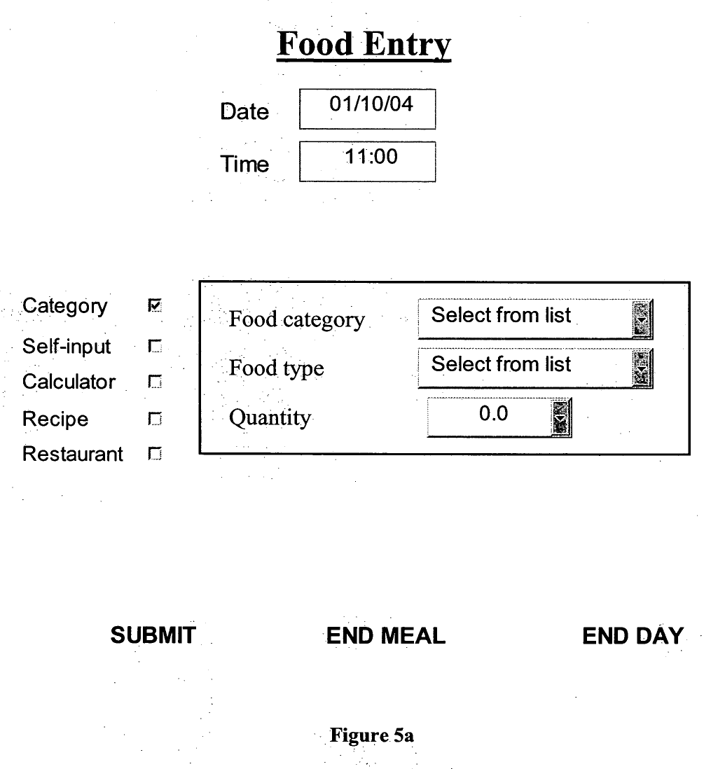

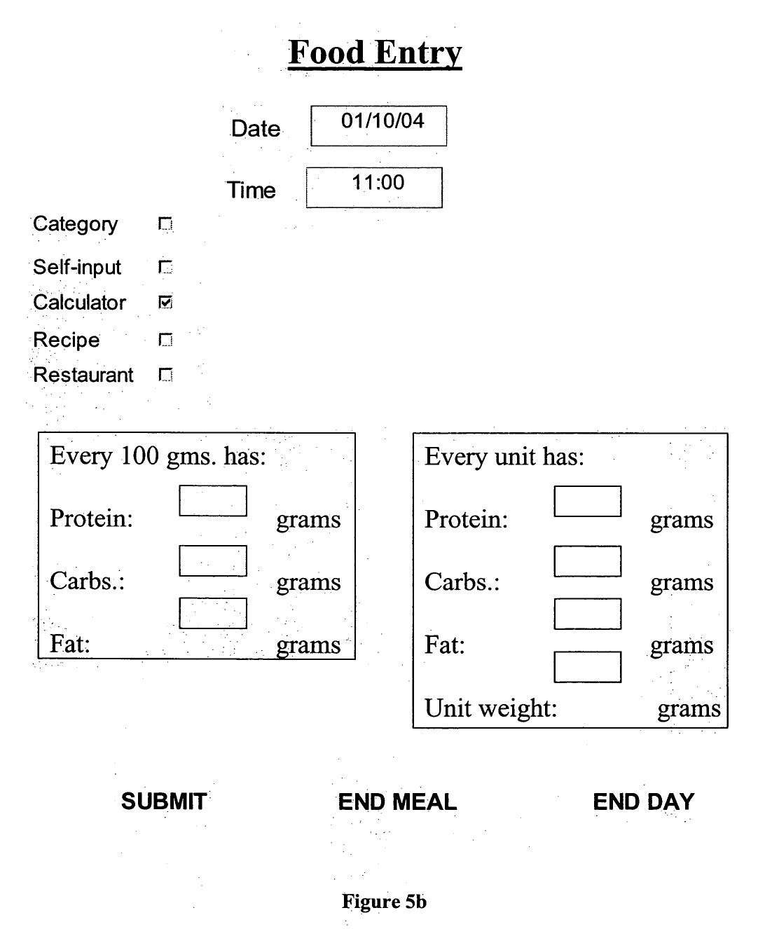

FIGS. 5a and 5b show examples of food-entry screens displayed to the user for the purpose of entering foods into the food diary. Each day, the user enters the date and time at which the food was eaten, and has five data input options, which he selects by checking the appropriate check box. The food entry then displays the appropriate fields for data input for the selected input option. In the Category option, shown in FIG. 5a, the user selects a type of food, a particular food of the selected type, from the appropriate drop-down lists, and a quantity of food eaten. In the Self-Entry mode (not shown), the user manually enters the amount of protein, carbohydrates and fat. In the Calculator mode, shown in FIG. 5b, the user manually enters the amount of protein, carbohydrates and fat (taken from a nutritional label on the food), either per unit or by weight. In the Recipe mode (not shown), for translating a specified recipe into quantities of carbohydrates, proteins, and fats. The user inputs a the number of eaters, and a list of ingredients and respective quantities each selected from the appropriate drop-down lists. The system translates the recipe to total amounts of carbohydrates, proteins and fats, and divides the totals by the number of eaters. (Similar data entry screens may be displayed to the user for purposes of user calculation of the nutritional content of a given food, without entering the selected food into the food diary.) After each food is entered, the user clicks the SUBMIT, the food is entered into the diary, and the food-entry screen is displayed again. At the end, the user clicks END MEAL or END DAY, as appropriate. After each food entry, the total portions of proteins, carbohydrates, and fats eaten during the current meal is displayed to the user, the number of portions eaten during the day, and the number of portions remaining for the rest of the day.

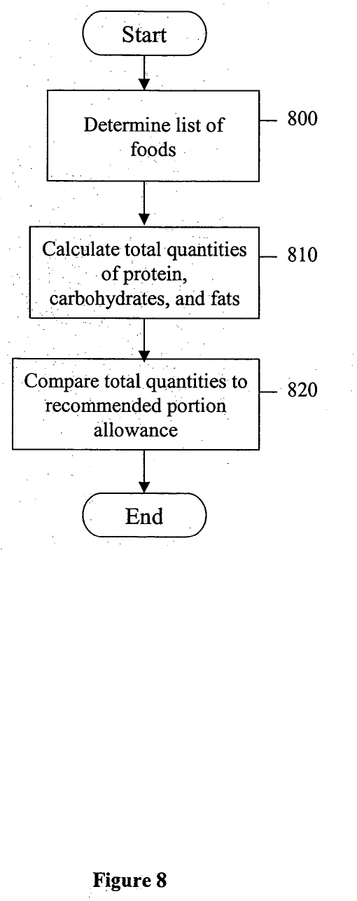

In step 130, the food consumption information received from the user is analyzed in accordance with the nutritional guidelines provided to the user in step 110. The purpose of the analysis is to track user adherence to the recommended nutritional regime, and to generate ongoing feedback and recommendations for the user. Preferably, the user also supplies information about daily physical activity, and the analysis is performed in light of the physical activity data. A preferred embodiment of the process of analyzing food consumption information is presented for FIG. 8 below.

In step 140, the results of the analysis are used to provide the user with dietary feedback. The feedback is provided to the user over the network. The dietary feedback serves as a teaching tool, whose purpose is to encourage and guide the patient into making the correct food choices. The feedback information preferably notifies the user of the remaining daily allowances of protein, carbohydrates, and fat. The feedback preferably also alerts the user to non-adherence to the nutritional guidelines and includes suggestions or improving eating patterns or daily menus, in order to encourage the user to stay within the recommended guidelines. For example, the feedback may include a comment that the user overate carbohydrates in a certain meal, finished his fats allowance for the day, ate too late or too soon, and so forth. The user feedback is preferably provided interactively, in response to a user query or after the user inputs food consumption information.

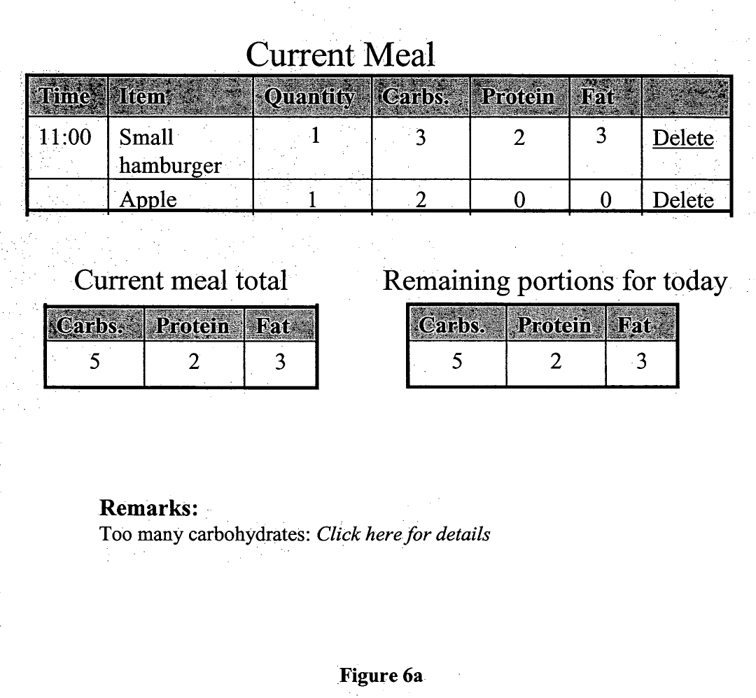

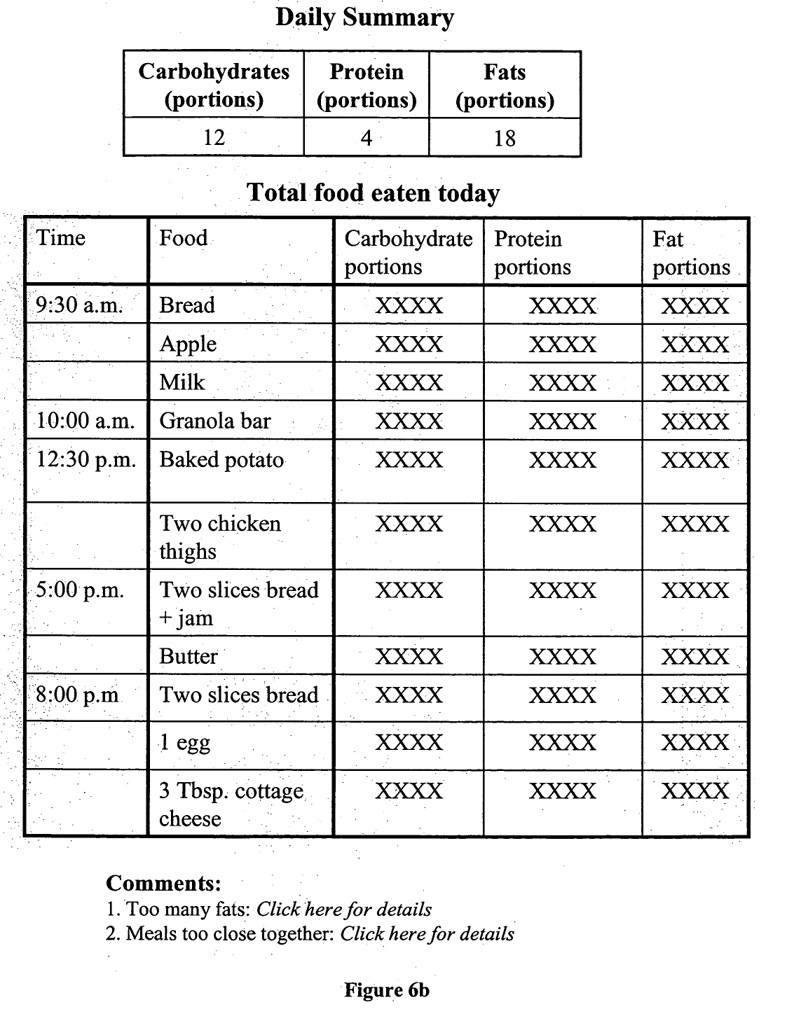

FIG. 6a show's a typical feedback screen which is displayed to the user after the user finishes entering food consumption information for a given meal. The feedback screen shows the translation of each food eaten during the meal to portions of carbohydrates, proteins and fats. The meal feedback information summarizes the total portions of carbohydrates, proteins and fats since the morning, and the number of portions remaining for the rest of the day, according to his personalized food pattern.

FIG. 6b shows a typical feedback screen which is displayed to the user at the end of the day. The daily feedback screen shows all the foods the user ate during the day, the distribution of each food item into nutritional components, and the total quantities (in portions) of each nutritional component consumed during the day. Suitable nutritional remarks are retrieved from a bank of nutritional remarks in accordance with the analysis performed in step 140, and provided to the user. The nutritional remarks may include a notification of a nutritional error (for example eating excessive carbohydrates), why is it wrong, and recommendations for fixin g the problem during the rest of the day or in the future. The user may also be periodically provided with nutritional remark statistics, indicating how frequently each nutritional remark has been output to the user. The statistics help the user to understand what improvements are needed to his diet, and describe the progress he has made nutritionally.

In the preferred embodiment, the user maintains a physical activity log, similar to the food database. The user enters the type and quantity of exercise performed during the current day, which is then analyzed and compared to the physical activity data used to calculate his nutritional guidelines in step 110, and whether the physical activity is of a desirable type and duration. The user is then provided with suitable physical activity remarks selected from a bank of physical activity remarks in accordance with the analysis. The user may also be provided with periodic comments, summarizing his physical activity over a certain time period.

In the preferred embodiment, the user inputs current personal information, which is preferably used to modify the nutritional guidelines. The nutritional guidelines are modified according to the current personal information and the previously-provided personal information. Thus, guidelines are established which take into account the user's current status, for example if he has changed his exercise routine, if he has finished the weight reduction stage and moved on to the weight maintenance stage, and so forth. After guideline modification, the nutritional feedback provided to the user is based on the modified guidelines.

Reference is now made to FIG. 7, which is a simplified flowchart of a method for establishing nutritional guidelines, according to a preferred embodiment of the present invention. In step 700, a recommended weight range is calculated for the user in accordance with the baseline height, weight, and age information provided by the user. In the preferred embodiment, the recommended weight range for men is calculated as follows:
Highest recommended weight=27*current weight BMI (2)
Lowest recommended weight=24*Current weight/BMI (3)
and the recommended weight range for women is calculated as follows:
Highest recommended weight=22*Current weight/BMI (4)
Lowest recommended weight=19*Current weight/BMI (5)

In step 710, a recommended caloric intake level is determined for the user according to: the user's gender, height, weight, age, physical activity level, and nutritional goals. The recommended caloric intake level is calculated by calculating the user's average daily calorie expenditure, and then adding or subtracting a given number of calories according to the user's nutritional goals, as described below.

The user's average daily calorie expenditure is determined from the user's Resting Metabolic Rate (RMR) and the average calories expended daily due to physical activity. The RMR is the rate at which an individual burns energy or calories at rest. The RMR is calculated according to the Harris-Benedict formula as:
RMR for Men=66+13.7*W+5*Height−6.8*Age (6)
RMR for Women=665+9.6*W+1.8*Height−4.7*Age (7)
where the user's height is entered in centimeters and age is in years. W is a measure of the user's weight in kilograms, which is determined by comparing the user's current weight to the weight range established by equations 2-5. If the user's current weight is within the weight range, W is taken as the user's current weight. However, if the user's current weight is outside the desired weight range, W is adjusted to take into account the difference in metabolic rates of fat and lean body tissue. Since fat tissue has one quarter of the resting metabolic rate (RMR) of lean body tissue, W is calculated as:
W=Highest recommended weight (kg.)+0.25*Fat overweight (kg.) (8)
where the highest recommended weight is defined for men and women in equations 2 and 4 respectively, and where Fat overweight is defined as:
Fat overweight=Current weight (kg.)−Highest recommended weight (kg.) (9)

Since, regular daily activity is estimated to enhance energy expenditure above the RMR by 20%, the RMR is increased by 20%.

The user's weekly energy expenditure from physical activity is determined by summing the caloric cost of all workouts performed during the week. The caloric cost of a given workout is determined by the kind of activity, its intensity, the duration of the exercise, and the user's body weight, and is calculated by methods known in the art. The user's weekly energy expenditure due to physical activity is calculated, and then a daily average is taken as:
Average energy expenditure=Weekly energy expenditure/7 (10)
The user's total daily energy expenditure (TEE) is thus:
TEE=RMR+Average physical activity energy expenditure (11)

The user's recommended energy intake, in calories, is then determined by adding or subtracting the number of calories necessary to achieve the user's nutritional goals. Preferably the recommended energy intake is given as:
1. Calorie intake for weight reduction=TEE−700 (to induce negative energy balance)
2. Calorie intake for maintaining a current weight=TEE−150 (to induce a small negative energy balance for small daily fluctuations)
3. Calorie intake for enhancing muscle mass=TEE+500
4. Calorie intake for pregnant women in the last trimester=TEE+500 (to enable extra energy expenditure for fetus development)
5. Calorie intake for lactating women TEE+500 (to enable extra energy expenditure for lactation)

In step 720, daily allowances of the nutritional components are determined from the recommended caloric intake level. The caloric budget is preferably distributed between proteins, carbohydrates, and fats components according to the following ratio: 60% carbohydrates, 15% proteins and 25% fats. The calorie allotment for each type of nutrient is then converted into grams, under the assumption that carbohydrates and proteins provide 4 calories per gram and fats provide 9 calories per gram. The daily allowances are preferably further translated into portions, according to the formulas given above.

The nutritional guidelines provided to the user (step 110 above) thus preferably consist of a daily calorie allowance distributed into specified amounts of proteins, carbohydrates, and fats.

Preferably, the nutritional recommendations further include a sample daily menu recommendation selected on the basis of the nutritional information in the foods database, thus assisting the user to translate the portions of carbohydrates, proteins and fats into quantified foods;

Preferably, the nutritional guidelines also include recommendations for health-related problems. A bank of health-related nutritional information and guidelines is stored in a memory, each one keyed to one or more health problems. When information received from the user includes notification of a health problem, the appropriate recommendation(s) are retrieved from the memory and output to the user.

Reference is now made to FIG. 8, which is a simplified flowchart of a method for analyzing food consumption information received from the user, according to a preferred embodiment of the present invention. In step 800, a list of foods (type and quantity) eaten by the user during a specified time period is generated, for example all foods eaten by the user from the beginning of the day to the current time. In step 810, the total quantities of each of the nutritional components contained in the listed foods are calculated. In step 820, each of the calculated totals is compared to the allowance specified in the user's nutritional guidelines. In a further preferred embodiment, an analysis of food consumption patterns, such as frequency of eating, timing of eating, and excessive quantities, is also performed.

In the preferred embodiment, the user's weight is tracked, and the user is provided with periodic comments. In order to track a real weight change, the user is queried for his weight after a period of at least 5 days since the previous weight entry. The system compares the user's weight change since the last measurement to the desired weight reduction for the same time period (0.5-1 Kg per week) as well as to the weight change expected according to food consumption and physical activity data received from the user. The user is provided with comments according to the change in weight—no reduction, an expected reduction, less than expected reduction etc. The comments may explain the reasons for the changes in the user's weight and guidelines for change, if needed., Similar tracking is preferably performed for the circumference of the user's waist, thighs, hips, and/or arms.

Preferably, the method contains the further step of billing the user. The nutritional counseling method is thus provided as a billable-service.

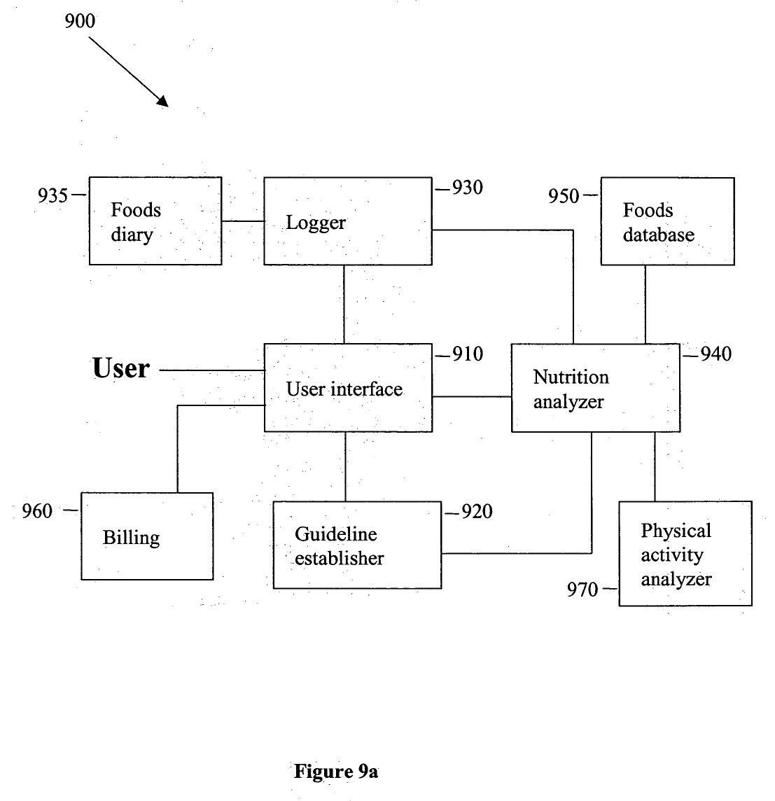

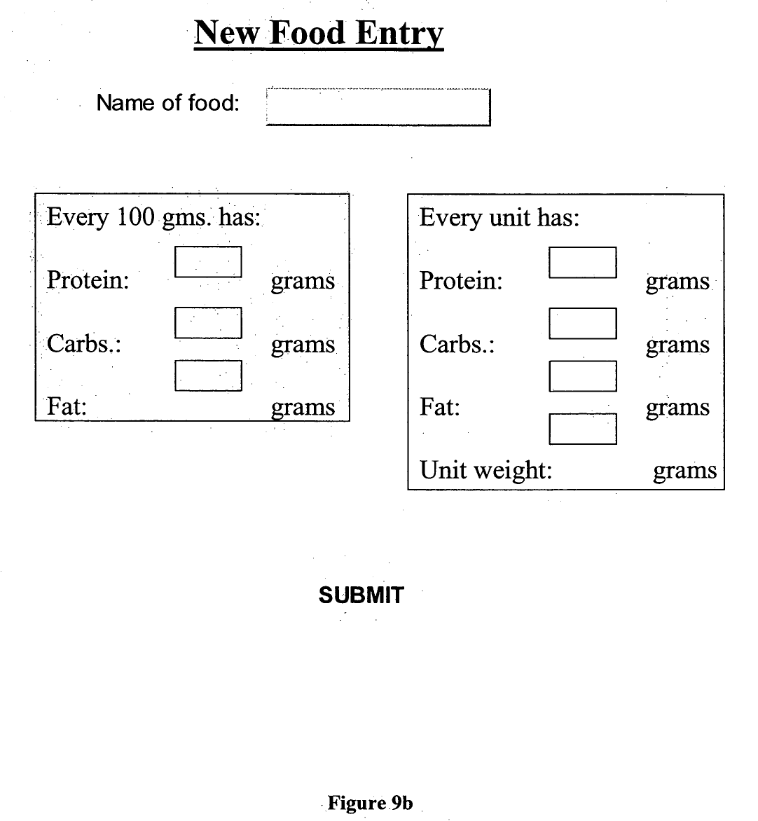

Reference is now made to FIG. 9a , which is a simplified block diagram of a nutritional counseling server, according to a preferred embodiment of the present invention. Nutritional counseling server 900 consists of user interface 910, guideline establisher 920, logger 930, and nutrition analyzer 940. User interface 910 serves for inputting and outputting data to a user. Guideline establisher 920 obtains personal information from the user and establishes nutritional guidelines for the user in accordance with the personal information, as described above. Guideline establisher 920 preferably also provides the user with physical activity guidelines. Logger 930 receives food consumption information from the user, and logs the food consumption information in food diary 935. Logger 930 preferably also records weight and/or physical activity information received from the user in food diary 935. Nutrition analyzer 940 compares the food consumption information to the nutritional guidelines, and provides dietary feedback to the user accordingly, as described above. The feedback provided to the user preferably consists of a recommended daily menu, recommended calorie intake, remaining calorie allowance, recommended allowances of nutritional components, remaining allowances of nutritional components, and/or nutritional error notification (along with suggestions for correcting the nutritional errors).

Preferably, in order to reduce the data storage requirements for the food diary, logger 930 replaces the detailed listing of foods recorded by the user during the current day with a record of the total portions of the nutritional components consumed during the course of the day at the end of each day.

In the preferred embodiment, nutritional counseling server 900 is interactive, and can provide immediate responses to user entries.

Preferably, user interface 910 connects to the user over a network. In the preferred embodiment, the connection to the user is made over one of: the Internet, a PSTN, a cellular network, a cable TV network, a satellite TV network, a LAN, a WAN, and MAN.

Preferably, nutritional counseling server 900 further contains foods database 950, which stores nutritional information for a multiplicity of foods, where the nutritional information preferably includes quantities of calories, proteins, carbohydrates, and fats in each food. Nutrition analyzer 940 may thus use the information in foods database 950 when calculating the amount of calories, etc. eaten by the user during a given time period. Foods database 950 is preferably updateable by the user to include nutritional information for a user-specified food. FIG. 9b shows an example of a new food entry screen, for user addition of a food to foods database 950.

Preferably, nutritional counseling server 900 contains billing functionality 960, which controls user access to the server.

Preferably, nutritional counseling server 900 further contains a physical activity analyzer 970, which calculates energy expenditure in accordance with physical activity information received from the user. Guideline establisher 920 and/or nutritional analyzer 940 may thus take the user's exercise regimen into account during their calculations.

In the preferred embodiment, logger 930 contains a meal entry unit for user input of the time of the meal, the foods eaten during the meal, and respective quantities of the foods eaten.

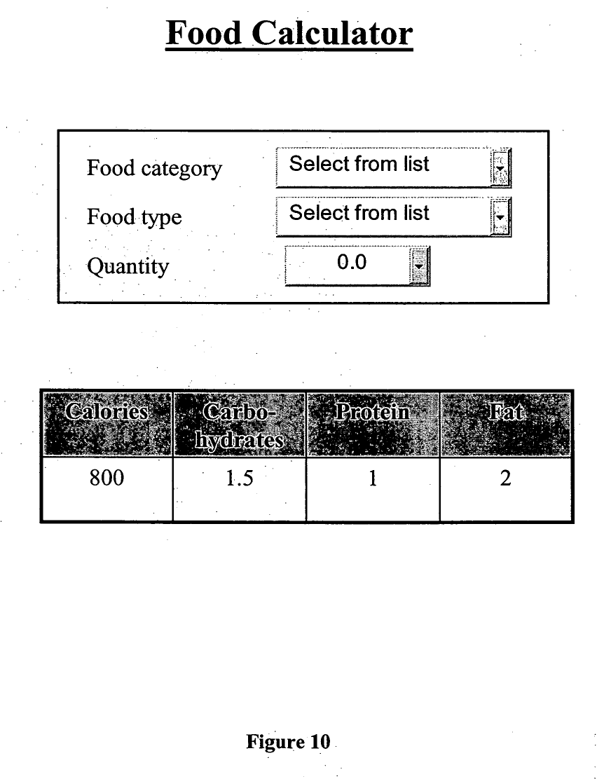

Preferably, nutritional counseling server 900 further contains a food calculator, for calculating the portions of foods, without entering the foods into the food diary. Data entry options preferably include one or more of the food entry options discussed in conjunction with FIGS. 5a and 5b. After the food data is input, the nutritional content of the food is preferably displayed to the user in portions, according to the portion definitions provided above. FIG. 10 shows an example of a data entry screen for the food calculator in Category mode.

For example, the food calculator may contain a recipe calculator, for translating a specified recipe into quantities of carbohydrates, proteins, and fats. The user inserts the recipe ingredients and the amount of each ingredient, and the system translates the recipe to total amounts of carbohydrates, proteins and fats, and divides the totals by the number of eaters.

Preferably, counseling server 900 further contains a menu planner, for providing a recommended menu in accordance with the guidelines. Preferably, nutritional counseling server further contains a favorite food list, selected from the foods database, which records the user's favorite foods, for printout or for incorporation into the recommended menus.

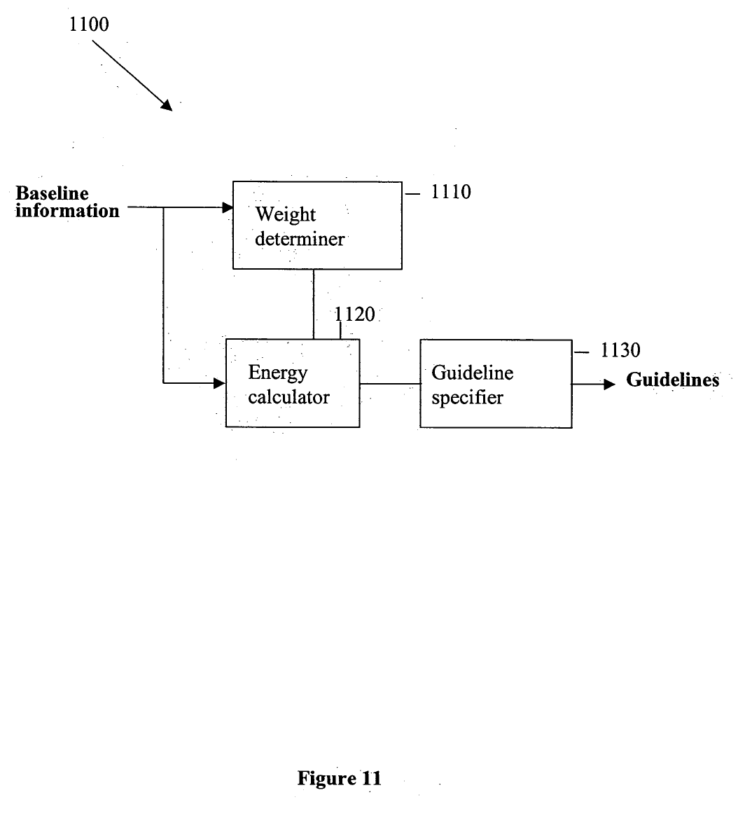

Reference is now made to FIG. 11, which is a simplified block diagram of a guidelines establisher, according to a preferred embodiment of the present invention. Guidelines establisher 1100 consists of weight determiner 1110, for calculating a recommended weight range in accordance with the user's gender, height, and age, energy calculator 1120, for determining a recommended caloric intake level in accordance with the user's gender, height, weight, age, physical activity level, and nutritional goals, and guideline specifier 1130, for determining a respective recommended allowance of nutritional components, in accordance with the recommended caloric intake level.

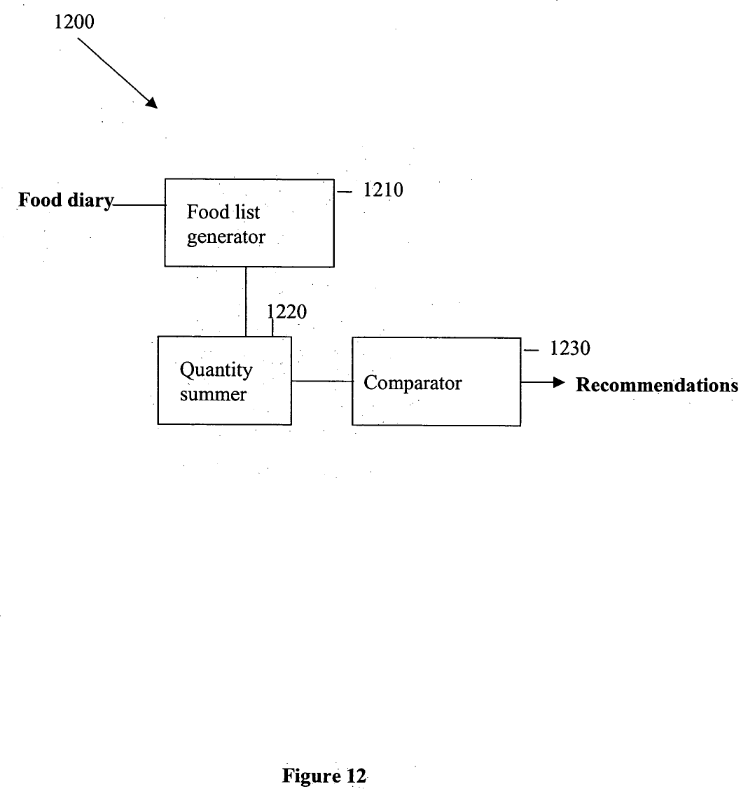

Reference is now made to FIG. 12, which is a simplified block diagram of a nutritional analyzer, according to a preferred embodiment of the present invention. Nutritional analyzer 1200 contains food list generator 1210, for retrieving a list of foods eaten by the user during a specified time period from the food diary, quantity summer 1220, for calculating the total quantities of nutritional components of the foods in the list, and, comparator 1230, for comparing the total quantities to the nutritional guidelines.

A further preferred embodiment consists of a computer-readable storage medium containing a set of instructions for nutritional counseling. The set of instructions contains a personal information input routine, a guideline establishment routine, a consumption information input routine, an analysis routine, and a feedback routine. The personal information input routine inputs personal information from the user. The guideline establishment routine establishes nutritional guidelines for the user in accordance with the personal information, and outputs the guidelines to the user. The food consumption input routine inputs food consumption information from the user. The analysis routine analyzes the received food consumption information in accordance with the guidelines. The feedback routine outputs dietary feedback to the user in accordance with the results of the analysis.

The abovedescribed embodiments provide a user with dietary assistance and support at all stages of the nutritional program. The program begins by providing the user with a dieatry plan, and continues to accompany the user on with day-to-day monitoring of his adherence to the dietary plan. Nutritional feedback, in particular interactive feedback, is a significant aspect of the present method, which allows the user to easily determine how well his is following the recommended regimen. The user may monitor his progress repeatedly during the day, to easily determine whether

The is eating correctly. The user is not obliged to maintain a mental or written record of food eaten during the day. The user also does not need to calculate whether he is eating properly or how much food he can eat for the remainder of the day in order to stick to his diet. The constant availability of nutritional counseling is also a psychological support to the user, who is provided with positive reinforcement for adherence to the recommended diet, and is alerted immediately of errors and problematic eating behaviors.

It is expected that during the life of this patent many relevant networks, methods for determining the nutritional composition of foods and/or caloric cost of physical activity, and nutritional components will be developed and the scope of the corresponding terms is intended to include all such new technologies a priori.

As used herein the term “about” refers to ±10%.20

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method for nutritional counseling, comprising:
allowing a user to connect remotely over a network;
receiving baseline personal information from said user over said network connection;
providing said user with nutritional guidelines in accordance with said personal information over said network connection;
receiving food consumption information from said user over said network connection;
analyzing said received food consumption information in accordance with said guidelines; and
providing dietary feedback to said user in, accordance with said analysis over said network connection.

2. A method according to claim 1, wherein said food consumption information comprises a type and quantity of food eaten, and the time and date the food was eaten.

3. A method according to claim 1, further comprising logging said food consumption information in a food diary.

4. A method according to claim 1, further comprising receiving current personal information from said user.

5. A method according to claim 4, further comprising modifying said nutritional guidelines in accordance with current and previously-provided personal information.

6. A method according to claim 1, wherein said guidelines are for maintaining a balance of nutritional components consumed by said user.

7. A method according to claim 6, wherein said nutritional components comprise proteins, carbohydrates, and fats.

8. A method according to claim 1, wherein said analyzing is in accordance with nutritional information stored for a plurality of foods in a foods database.

9. A method according to claim 8, further comprising updating said foods database to include nutritional information for a user-specified food.

10. A method: according to claim 1, wherein said personal information comprises said user's gender, age, current weight, height, and nutritional goals.

11. A method according to claim 10, wherein said personal information further comprises said user's physical activity regimen.

12. A method according to claim 10, further comprising providing said user with physical activity guidelines in accordance with said personal information, over said network connection.

13. A method according to claim 10, wherein said personal information further comprises said user's health status.

14. A method according to claim 1, wherein establishing nutritional guidelines comprises:
calculating a recommended weight range in accordance with said user's gender, height, and age;
determining a recommended caloric intake level in accordance with said user's gender, height, weight, age, physical activity level, and nutritional goals; and
determining respective allowances of proteins, carbohydrates, and fats, in accordance with said recommended caloric intake level.

15. A method according to claim 2, wherein said analyzing comprises:
determining a list of foods eaten by said user during a specified time period;
calculating the total quantities of carbohydrates, proteins, and fats for foods on said list; and
comparing said total quantities to said nutritional guidelines.

16. A method according to claim 6, wherein said feedback comprises remaining daily allowances of said nutritional components.

17. A method according to claim 1, wherein said feedback comprises alerting said user to non-adherence to the guidelines.

18. A method according to claim 1, wherein said feedback comprises nutritional error notification, and suggestions for correcting said nutritional errors.

19. A method according to claim 1, wherein said network comprises at least one of: the Internet, a Public Switched Telephone Network (PSTN), a cellular telephone network, a cable TV network, a satellite TV network, a local-area network (LAN), a wide-area network (WAN), and a metropolitan-area network (MAN).

20. A method according to claim 1, further comprising providing said user with a recommended menu.

21. A method according to claim 1, wherein said analyzing is in accordance with physical activity information received from said user.

22. A method according to claim 21, further comprising monitoring a user's physical activity, in comparison with a physical activity level provided with said personal information.

23. A method according to claim 21, further comprising monitoring a user's physical activity, in comparison with physical activity guidelines provided to said user.

24. A method according to claim 1, further comprising monitoring a user weight in accordance with weight information received from said user.

25. A method according to claim 1, further comprising comparing said user weight to an expected user weight.

26. A method according to claim 1, further comprising calculating the portions of carbohydrates, proteins, and fats present in a specified food type and quantity.

27. A method according to claim 1, further comprising receiving a recipe comprising a listing of types of foods and respective quantities, and translating said recipe into quantities of carbohydrates, proteins, and fats.

28. A method according to claim 1, further comprising billing said user.

29. A nutritional counseling server, comprising:
a user interface, for interfacing to a user over a network;
a guideline establisher associated with said user interface, for obtaining baseline personal information from said user and establishing nutritional guidelines for said user in accordance with said personal information;
a logger associated with said user interface, for receiving food consumption information received from said user and logging said food consumption information in a food diary; and
a nutrition analyzer associated with said user interface and said logger, for analyzing said food consumption information according to said guidelines, and providing dietary feedback to said user in accordance with said analysis.

30. A nutritional counseling server according to claim 29, further comprising a foods database associated with said nutrition analyzer, for storing nutritional information for a plurality of foods.

31. A nutritional counseling server according to claim 30, wherein said foods database is updateable by said user to include nutritional information for a user-specified food.

32. A nutritional counseling server according to claim 29, wherein said guidelines establisher comprises:
a weight determiner, for calculating a recommended weight range in accordance with said user's gender, height, and age;
an energy calculator, for determining a recommended caloric intake level in accordance with said user's gender, height, weight, age, physical activity level, and nutritional goals; and
a guideline specifier associated with said weight determiner and said energy calculator, for determining a respective recommended allowance of proteins, carbohydrates, and fats, in accordance with said recommended caloric intake level.

33. A nutritional counseling server according to claim 32, wherein said nutritional analyzer comprises:
a food list generator, for retrieving a list of foods eaten by said user during a specified time period from said food diary;
a quantity summer associated with said food list generator, for calculating the total quantities of carbohydrates, proteins, and fats for said list of foods; and
a comparator associated with said quantity summer, for comparing said total quantities to said nutritional guidelines.

34. A nutritional counseling server according to claim 29, wherein said feedback comprises at least one of the following types of recommendations: a recommended menu, a recommended daily allowance of said nutritional components, a remaining daily allowance of said nutritional components, nutritional error notification, and suggestions for correcting nutritional errors.

35. A nutritional counseling server according to claim 29, further comprising a physical activity analyzer associated with said nutrition analyzer, for calculating energy expenditure in accordance with physical activity information received from said user.

36. A nutritional counseling server according to claim 29, wherein said nutrition analyzer is operable to provide interactive responses to user entries.

37. A nutritional counseling server according to claim 29, wherein said network comprises at least one of: the Internet, a PSTN, a cellular telephone network, a cable TV network, a satellite TV network, a LAN, a WAN, and a MAN.

38. A nutritional counseling server according to claim 30, further comprising a menu planner associated with said foods database, for providing a recommended menu in accordance with said guidelines.

39. A nutritional counseling server according to claim 29, wherein said logger is further operable to record weight information received from said user in said diary.

40. A nutritional counseling server according to claim 29, wherein said logger comprises a meal entry unit for user entry of a meal time, foods eaten during said meal, and respective quantities of said foods.

41. A nutritional counseling server according to claim 29, further comprising a food calculator, for calculating quantities of carbohydrates, proteins, and fats present in a specified food type and quantity.

42. A nutritional counseling server according to claim 41, wherein said food calculator comprises a recipe calculator, for translating a specified recipe into quantities of carbohydrates, proteins, and fats.

43. A nutritional counseling server according to claim 29, further comprising billing functionality.

44. A nutritional counseling server according to claim 38, further comprising a favorite food list for recording said user's favorite foods for incorporation into a recommended menu.

45. A computer-readable storage medium containing a set of instructions for nutritional counseling, comprising:
a personal information input routine, for inputting personal information from a user;
a guideline establishment routine, for establishing nutritional guidelines for said user in accordance with said personal information and outputting said guidelines to said user;
a consumption information input routine, for inputting food consumption information from said user;
an analysis routine, for analyzing said received food consumption information in accordance with said guidelines; and
a feedback routine for outputting dietary feedback information to said user in accordance with the results of said analysis.
